(19) United States
(12) Patent Application Publication
(10) Pub. No.: US 2004/0097468 A1
(43) Pub. Date: May 20, 2004

(54) Method of treating osteoporosis and other bone disorders with upfront loading of bisphosphonates, and kits for such treatment

(76) Inventor: Sunil J. Wimalawansa, East Brunswick, NJ (US)

Correspondence Address: Sunil J. Wimalawansa, MD, Ph.D, FRCP, Robert Wood Johnson Medical School, MEB - 372, P.O. Box 19, New Brunswick, NJ, 08903-0019, US

(21) Appl. No.: 10/299,975

(22) Filed: Nov. 20, 2002

(51) Int. Cl. A61K 31/675; A61K 31/66

(52) U.S. Cl. 514/89; 514/102

(57) ABSTRACT

Administering thereto-upfront loading of a bisphosphonate agent can be used to treat primary and secondary osteoporosis, other metabolic bone diseases, alleviation of bone pain, transplant and drug-induced bone loss, Paget's disease of bone, loosening of prosthesis, or metastatic bone diseases in mammals, preferably a human female or a male. A bisphosphonate drug can be administered as a loading dose upfront. Bisphosphonates can be administered by themselves or combined with, one or more other medications acting on bone, such as HRT, selective estrogen receptor modulating drug, calcitonin, parathyroid hormone, fluoride, androgen, sex-steroid hormone analogues, nitroglycerin growth factors and their analogs, peptides and proteins and their analogues, or any other novel therapeutic agents.

This new regimen of administration of an anti-osteoporosis drug (e.g., a bisphosphonate) by itself, or in combination with other medications, can be used in mammals, preferably human (in women and men) for prevention and treatment of osteoporosis (e.g., postmenopausal, glucocorticoid- or drug-induced osteoporosis and osteoporosis in men, etc.) and other metabolic bone disorders, metastatic bone disease, transplant bone disease, Paget's disease, and prevention and treatment of loosening of prosthesis. Disclosed are methods for rapid inhibition of bone resorption in mammals while obtaining a rapid reduction of bone turnover and biomarkers, rapid increase of bone mineral density, and rapid reduction of fractures. Also disclosed are pharmaceutical compositions and kits for carrying out the therapeutic methods disclosed herein.

### BACKGROUND OF THE INVENTION

A variety of disorders in humans and other mammals involve, or are associated with, abnormal bone resorption. Such disorders include, but are not limited to, osteoporosis, Paget's disease, periprosthetic bone loss or osteolysis, and hypercalcemia of malignancy. The most common of these disorders is osteoporosis, which, in its most frequent manifestation, occurs, in postmenopausal women. Osteoporosis is a systemic skeletal disease characterized by a low bone mass and micro architectural deterioration of bone tissue with a consequent increase in bone fragility and susceptibility to fracture. Osteoporosis, as well as other disorders associated with bone loss, Paget's disease of bone, and metabolic or metastatic bone diseases require therapies to be delivered to the site (i.e., to the bone) quickly, and not over a course of several months, as with the standard methods of delivery and application with bisphosphonates (whether these are delivered orally or intravenously). However, these agents take several months to get adequate cumulative dose absorption to be effective at the skeleton.

Multinucleated cells called osteoclasts are responsible for causing bone loss through a process known as bone resorption. It is well known that bisphosphonates are selective inhibitors of osteoclastic bone resorption, making these compounds important therapeutic agents in the treatment or prevention of a variety of generalized or localized bone disorders caused by, or associated with, abnormal bone resorption. See H. Fleisch, Bisphosphonates In Bone Disease, From The Laboratory To The Patient, 2nd Edition, Parthenon Publishing (1995), which is incorporated by reference herein in its entirety.

At present, a great amount of pre-clinical and clinical data exists for the potent bisphosphonate compounds such as alendronate, risedronate, and other bisphosphonates. Evidence suggests that other bisphosphonates, such as risedronate, tiludronate, ibandronate and zolendronate, have many common properties, including high potency as inhibitors of osteoclastic bone resorption. An older bisphosphonate compound, etidronate, also inhibits bone resorption. However, unlike the more potent bisphosphonates, etidronate impairs mineralization at doses used clinically and may give rise to osteomalacia, a condition resulting in an undesirable decrease in bone mineralization. See Boyce, B. F., Fogelman, I., Ralston, S. et al. (1984) Lancet 1(8381), pp. 821-824 (1984), and Gibbs, C. J., Aaron, J. E.; Peacock, M. (1986) Br. Med. J. 292, pp. 1227-1229 (1986), Wimalawansa, S. J., Combined therapy with oestrogen and etidronate has an additive effect on the bone mineral density: four-year prospective study. American Medical Journal, 99: 36-42, 1995, all three of which are incorporated by reference herein in their entirety.

This invention relates to a new method of administering bone-acting medications; in particular, a bisphosphonate for prevention and treatment of osteoporosis and other bone mineral disorders, and prevention of bone loss and fractures. This invention is also an encompensate-loading dose of a bisphosphonate administered together with an estrogen and/or a progestin, calcitonin, SERMs, PTH; cytokine, protein or peptide analogue, growth factors or their analogues; vitamin D or its analogues; any bone acting anabolic agents, nitroglycerin, or any other drug acting on the skeletal system. Some compounds and methods of application are also useful in men to decrease bone turnover, decrease fractures, and hence prevention and treatment of osteoporosis, and for treatment of other metabolic bone disorders.

Bisphosphonates can be administered in combination with hormone replacement therapy or with other drugs acting on the skeleton. Wimalawansa S. J. Combined therapy with oestrogen and etidronate has an additive effect on the bone mineral density: four-year prospective study. American Medical Journal, 99: (1995): 36-42; Wimalawansa S. J. Four-year randomized controlled trial of hormone replacement therapy and bisphosphonate alone or in combination in women with postmenopausal osteoporosis. American Journal of Medicine, 104: (1998): 219-226; Wimalawansa S. J. Prevention and treatment of osteoporosis: Efficacy of combined therapies of hormone replacement therapy with anti-resorptive agents. Journal of Clinical Densitometry, 3: (2000): 1-15, 2000: Lindsay R, Cosman F, Lobo R A, Walsh B W, Harris S T, Regan J E, Liss C L, Melton M E, Byrnes C A. Addition of alendronate to ongoing hormone replacement therapy in the treatment of osteoporosis: A randomize, controlled clinical trial. J Clin Endocrinol Metab 84: (2000): 3076-3081; Cosman F, Neives J, Woelfert L, Shen V, Lindsay R. Alendronate does not block the anabolic effect of PTH in postmenopausal osteoporotic women. J Bone Min Res 13: (1998):1051-1055, all five of which are incorporated by reference herein in their entirety.

This invention is also applicable to both primary and secondary osteoporosis in all mammals, preferably human. In both sexes, for the secondary osteoporosis, the underlying causative factors are numerous, including medication-induced osteoporosis (e.g., corticosteroids, antiepileptics, anticoagulants, thyroxin, etc.), immunosuppressant agents used in prevention of graft rejection and other disorders (prednisolone, cyclosporin, FK506), malignancies (e.g., multiple myeloma), transplant- and drug-induced bone loss, immobilization- and weightlessness-induced bone loss and other conditions of bone loss.

The category of metabolic bone disease also includes osteoporosis and Paget's disease. Osteoporosis is the most common disorder associated with aging. More than 1.5 million Americans have fractures related to osteoporosis each year with attendant pain, deformity and loss of independence. The annual cost to the U.S. health care system is at least $18 billion. Wimalawansa S J. Osteoporosis: Time to act (hand-book for the primary care physicians), 2002, incorporated by reference herein in their entirety. Because of the aging of the population and increases over time in the incidence of fractures, these costs will more than double over the next 30 years unless a comprehensive program of prevention and treatment is initiated soon.

The most important preventable cause of fractures is low bone mass. During the course of their lifetimes, women lose about 50 percent of their cancellous bone and 35 percent of their cortical bone and men lose about 30 percent and 25 percent, respectively. Cancellous bone is predominantly present in the spinal vertebrae and at the ends of long bones; these areas are the main sites of osteoporotic fractures. The tendency of the elderly to fall, however, is an important independent cause of fractures. Although little can be done at present to prevent such falls, important advances have been made in methods of retarding bone loss. However, all these methods take several months to a year or more before having an impact on the bone and fracture reduction. A loading dose of bisphosphonate is designed to rapidly deliver the agent of interest (e.g., a bisphosphonates) to bone and thereby expedite the effects on decrease of bone turnover, increased BMD, and bone fractures.

Rationale:

Bisphosphonates are highly effective in enhancing the BMD and decreasing the fracture rates. However, the absorption of bisphosphonates is usually less than 1% of that of the orally administered dose (e.g., alendronate, risedronate, etidronate, tiludronate, etc.). Therefore, generally it may take several months or up to a year or more to get an adequate amount of a bisphosphonate into bone to be effective in the reduction of bone turnover and fracture reduction. This is also true for the “standard doses” of bisphosphonate administered intravenously (pamidronate, ibandronate and zolendronate, etc.). It was hypothesized that if we could get an adequate amount of the bisphosphonate into bone earlier in the course (effective amounts to rapidly decrease bone turnover) of a treatment, then one should see a rapid response and also enhance the beneficial effects on BMD and fracture reduction.

The rationale of using loading doses of bisphosphonates is to start therapy with initial large doses in order to get the drug incorporated into the bone rapidly. This novel approach results in more rapid changes in both biochemical markers of bone turnover and in bone mineral density than is seen with conventional dosage regimens. It should significantly enhance fracture reduction efficacy and would be particularly beneficial for patients with a high fracture risk and those with established osteoporosis.

Bisphosphonates are widely used in the treatment of osteoporosis and daily or once a week administration of alendronate or risedronate decrease the rate of bone turnover, increase BMD, and reduce the risk of vertebral and non-vertebral fractures including those of the hip. But these will take several months or years to be effective. On the basis of principles of bisphosphonate pharmacology and bone remodeling, a weekly regimen providing a bisphosphonate dose equal to the total of seven daily doses was developed for alendronate and for risedronate. These weekly or infrequent administrations of bisphosphonates over longer periods have the same efficacy as their respective daily regimens, but no beneficial expedited effects on bone have yet been reported.

Despite their therapeutic benefits, bisphosphonates are poorly absorbed from the gastrointestinal tract [see B. J. Gertz et al., Clinical Pharmacology of Alendronate Sodium, Osteoporosis Int., Suppl. 3: S13-16 (1993) and B. J. Gertz et al., Studies of the oral bio-availability of alendronate, Clinical Pharmacology and Therapeutics, vol. 58, number 3, pp. 288-298 (1995), which are incorporated by reference herein in their entirety. Intravenous administration has been used to overcome this bioavailability problem.

If oral administration of the loading dose of a bisphosphonate is desired, relatively high doses can be administered to compensate for the low bioavailability from the gastrointestinal tract. To improve this low bioavailability, it is generally recommended that the patient take the bisphosphonate on an empty stomach and fast for at least 30 minutes afterwards. In spite of this, absorption from an oral does of a bisphosphonate is under 1% in general. However, many patients find the need for such fasting on a daily basis to be inconvenient. Some bisphosphonates, in particular amino groups containing bisphosphonate such as orally administered pamidronate, have been associated with esophageal ulcers. See E. G. Lufkin et al., Pamidronate: An Unrecognized Problem in Gastrointestinal Tolerability, Osteoporosis International, 4: 320-322,1994, which is incorporated by reference herein in its entirety.

Although not as common, the use of alendronate and risedronate has also been associated with esophagitis and/or esophageal ulcers. See P. C. De Groen, et al., Esophagitis Associated With The Use Of Alendronate, New England Journal of Medicine, vol. 335, no. 124, pp. 1016-1021, 1996, D. O. Castell, Pill Esophagitis—The Case of Alendronate, New England Journal of Medicine, vol. 335, no. 124, pp. 1058-1059, 1996, and U. A. Liberman et al., Esophagitis and Alendronate, New England Journal of Medicine, vol. 335, no. 124, pp. 1069-1070 (1996), which are incorporated by reference herein in their entirety. The degree of adverse gastrointestinal effects of bisphosphonates may occur independently of the dose or even the frequency of administration. See C. H. Chestnut et al., Alendronate Treatment of the Postmenopausal Osteoporotic Woman: Effect of Multiple Dosages on Bone Mass and Bone Remodeling, The American Journal of Medicine, vol. 99, pp. 144-152, 1995, which is incorporated by reference herein in its entirety. Also, these adverse esophageal effects appear to be more prevalent in patients who do not take the bisphosphonate with an adequate amount of liquid or who lie down shortly after dosing, thereby increasing the chance for esophageal reflux. So correct dosing procedure is important with any of the bisphosphonates, but especially with higher doses.

Current oral bisphosphonate therapies generally fall into two categories: (1) those therapies utilizing continuous daily treatment, and (2) those therapies utilizing a cyclic regimen of treatment and rest periods. The standard continuous daily, several times a week, once a week or once every few weeks treatment regimens normally involve the chronic administration of relatively low doses of the bisphosphonate compound with the objective of delivering the desired cumulative therapeutic dose over the course of the treatment period which usually takes months to years. To build up therapeutically adequate levels of bisphosphonates in the skeleton usually takes over many months to a year or more. However, a loading dose of a bisphosphonate as this invention described (i.e., at high doses initially) will get the required adequate amount of the agent bisphosphonate into the skeleton in a very short time period (i.e., within days). This will then allow a rapid action of this agent on bone to decrease bone turnover, increase BMD, and decrease fracture rates. Furthermore, this will allow preservation and reconstruction of the micro architecture of bone, which will have major impact on the bone strength and decreasing fractures.

Cyclic treatment regimens were developed because some bisphosphonates, such as etidronate, when given daily for more than several days, have the disadvantage of actually causing a decline in bone mineralization, i.e., osteomalacia. U.S. Pat. No. 4,761,406, to Flora et al, issued Aug. 2, 1988, which is incorporated by reference herein in its entirety, describes a cyclic regimen developed in an attempt to minimize the decline in bone mineralization while still providing a therapeutic anti-resorptive effect. Generally, cyclic regimens are characterized as being intermittent, as opposed to continuous treatment regimens, and both have treatment periods during which the bisphosphonate is administered and non treatment periods to permit the systemic level of the bisphosphonate to return to baseline. However, the cyclic regimens, relative to continuous dosing (daily or weekly), appear to result in a decreased therapeutic antiresorptive efficacy. Data on risedronate suggests that cyclic dosing is actually less effective than continuous daily dosing for maximizing antiresorptive bone effects. See L. Mortensen, et al., Prevention Of Early Postmenopausal Bone Loss By Risedronate, Journal of Bone and Mineral Research, vol. 10, supp. 1, p. s140, 1995, which is incorporated by reference herein in its entirety. The cyclic regimens are cumbersome to administer and have the disadvantage of lower patient compliance and consequently compromised therapeutic efficacy. U.S. Pat. No. 5,366,965, to Strein, issued Nov. 22, 1994, which is incorporated by reference herein in its entirety, attempts to address the problem of adverse gastrointestinal effects by administering a polyphosphonate compound, either orally, subcutaneously, or intravenously, according to an intermittent dosing schedule, having both a bone resorption inhibition period and a no-treatment rest period. However, the regimen has the disadvantage of not being continuous and regular and requires nontreatment periods ranging from 20 to 120 days. PCT Application No. WO 95/30421, to Goodship et al, published Nov. 16, 1995, which is incorporated by reference herein in its entirety, discloses methods for preventing prosthetic loosening and migration using various bisphosphonate compounds.

Any of the available pharmaceutical preparations of bisphosphonates can be combined in this fashion. For example, disodium etidronate 5-10 mg/kg body weight for 14 days, in every 90 days; alendronate 10 mg, once daily or 70 mg once a week; risedronate 5 mg daily or 35 mg once a week; and other preparations such as pamidronate, or clodronate, and any other bone acting agents. Calcitonin: any of the available pharmaceutical preparations of calcitonin, i.e. salmon, eel or human calcitonin, dosage range 10-400 i.u., daily, enteral, parenteral or nasal route. SERM; raloxifene 60 mg once a day. Anabolic steroids: Any of the available pharmaceutical preparations of anabolic steroids, i.e. Nandrolone decanoate 50 mg. im injection every 3-4 weeks. Androgens: Any of the available pharmaceutical preparations of Testosterone (50-200 mg, once in 3-4 weeks) or its analogs, or daily, twice a week or once a week administration of patches, gel or cream containing testosterone or its analogues. Fluoride: Any of the available pharmaceutical preparations of fluoride, e.g., sodium fluoride, 5-50 mg, once daily, Parathyroid hormone or its fragments (20 microgram daily for 12-24 months), or equivalents of PTH or PTH-rp analogues.

The pharmacologically active agents employed in this invention can be administered in a mixture with conventional excipients, i.e., pharmaceutically acceptable liquid, semi-liquid or solid organic or inorganic carriers suitable, e.g., for parenteral or enteral application and which do not deleteriously react with the active compound in a mixture therewith. Suitable pharmaceutically acceptable carriers include, but are not limited to, water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxy methylcellulose, and polyvinyl pyrrolidone, etc.

The pharmaceutical preparations can be sterilized, and if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

Suitable for oral administration are inter alia, tablets, liquids, dragees, capsules, pills, granules, suspensions and solutions. Each unit dose, e.g., each tablespoon of liquid or each tablet, or dragee contains, for example, 0.001-1000 mg of each active agent.

Solutions for parenteral administration contain, for example, 0.01-1% of each active agent in an aqueous or alcoholic solution. For parenteral application, particularly suitable are solutions, preferably oily or aqueous, as well as suspensions, emulsions, depot preparations or implants, including suppositories, creams, ointments, and transdermal patches.

In a preferred aspect, the composition of this invention is adapted for ingestion. For enteral application, particularly suitable are unit dosage forms, e.g., tablets, liquids, dragees or capsules having talc and/or a carbohydrate carrier or binder of the like.

The carriers preferred are lactose and/or cornstarch and/or potato starch; particulate solids, e.g., granules; and liquids and semi-liquids, e.g., syrups and elixirs or the like, wherein a sweetened vehicle is employed. Sustained release compositions can be formulated, including those wherein the active compound is protected with differentially degradable coatings, e.g., by microencapsulation, multiple coatings, etc.

There have been previous attempts to give bisphosphonates parenterally at infrequent intervals to increase adherence to treatment and to avoid the gastrointestinal toxicity that may accompany the daily oral use. These include intravenous infusions of pamidronate 30-60 mg, alendronate 10 mg, and intravenous injections of ibandronate up to 2 mg every 3 months, or zolendronate (also identified as zolondronic acid) administered 1 mg every 4 months, 2 mg every 6 month, or 4-5 mg once a year. These regimens generally all decreased the rate of bone resorption and increased BMD to levels similar to those achieved by daily oral bisphosphonate leading, in some cases, to off label use of intravenous bisphosphonates in the management of osteoporotic patients. However, the first placebo-controlled study with intravenous bisphosphonate ibandronate and fracture endpoints did not meet expectations. Intravenous ibandronate injections, 0.5 or 1.0 mg every 3 months for 3 years induced a moderate increase in BMD and a small, but not significant, decrease in the incidence of new osteoporotic fractures; this is clearly due to inadequate dosing. Due to the delivery of inadequate doses via parenteral rout, none of these agents administered parenterally (intravenous, dermal, etc.) has shown to decrease fracture rates.

We have examined the effectiveness of loading doses of oral and intravenously administered bisphosphonates on BMD in comparison with the standard doses of the same agent. We have compared the effects of loading doses of alendronate, risedronate and pamidronate with their standard recommended doses for osteoporosis over a one-year period in humans. Results demonstrated a significant increase of BMD in both oral and intravenously administered agents within 3-6 months (with the biomarker changes observed within the first 1-6 weeks) in comparison with the 9-12 months for the standard therapy.

Since the fracture efficacy of an anti-resorptive drug depends on the rapidity of the reduction of bone turnover and enhancement of the BMD, this new approach of administration of bisphosphonates (i.e., loading doses) should rapidly reduce fracture rates in comparison with the standard recommended regimens. Furthermore, this mode of administration with front-loading of bisphosphonates will decrease the bone turnover within days to weeks and reduces fracture in weeks instead of months to years in comparison with the traditional well accepted and FDA-approved regimens. Since 1 in 5 patients with a fracture re-fracture within a year, it is critical to make these drugs work within a short period of time; the new regimen described in this application delivers this. This approach of therapy should be particularly beneficial for those patients with high bone turnover and those with established osteoporosis who require rapid effects.

Prevention and Treatment of Osteoporosis:

One aspect of the present invention provides a method for the rapidly prevent and treatment of primary and secondary osteoporosis, including medication-induced osteoporosis (i.e. corticosteroid-induced osteoporosis, transplant-bone disease) and other metabolic bone disorders with a loading dose of a bisphosphonate.

It is a further objective to provide a method for the rapid prevention and treatment of osteoporosis and other metabolic bone disorders using an estrogenic agent or any bone-acting agent in combination with a loading dose of a bisphosphonate.

Another objective is to provide a method of rapid prevention and treatment of male primary and secondary osteoporosis and other metabolic bone disorders using a loading dose of a bisphosphonate alone, or together with another bone-effective agent.

A further objective is the provision of pharmaceutical compositions useful in practicing the methods of this invention using a method of administration. Other objectives will be apparent to those who are skilled in the art to which this invention pertains.

Another objective is to provide a method of prevention and treatment of metabolic bone diseases and other bone disorders such as Paget's disease of bone and osteogenesis imperfecta using a loading dose of a bisphosphonate.

Another objective is to provide a method of prevention and treatment of metastatic bone disease and prevention and treatment of loosening of orthopedic prosthesis using a loading dose of a bisphosphonate.

Another objective is to provide a method of prevention or treatment of cord compression secondary to vertebral fractures as a consequence of osteoporosis, orthopedic surgery, metastatic bone disease, or Paget's disease of bone using a loading dose of a bisphosphonate.

Another objective is to provide a method of prevention and treatment of hypercalcemia of malignancy by using a loading dose of a bisphosphonate.

Another objective is to provide a method of prevention and treatment of bone loss associated with microgravity, bed-ridden conditions such as following a stroke or a fracture, or for astronauts in long-duration space flights.

Another objective is to provide a method of reliving pain associated with any bone disease using a loading dose of a bisphosphonate.

Disclosed is a therapy protocol for treating patients having osteoporosis, Paget's disease of bone, metastatic bone disease, malignancy hypercalcemia or metabolic bone disease by administering an intravenous, parenteral, or oral loading dose of a bisphosphonate with an appropriate continuation of therapy.

It is an objective of the present invention to provide a rapid method for inhibiting bone resorption and the conditions associated therewith.

It is another objective of the present invention to provide methods for rapidly treating abnormal bone resorption (i.e., turnover), and the conditions associated therewith.

It is another objective of the present invention to provide methods for rapidly preventing abnormal bone resorption, turnover, and the conditions associated therewith.

It is another objective of the present invention to provide methods for rapidly preventing fractures and the conditions associated therewith.

It is another objective of the present invention to provide methods, which are oral, parenteral, or intravenous.

It is another objective of the present invention to provide such methods in mammals, especially in human males and females.

It is another objective of the present invention to provide such methods in mammals, especially in humans, using a kit.

It is another objective of the present invention to provide such methods comprising a continuous loading dosing schedule having a loading dose administered orally periodically ranging from daily, every other day, or 2-5 times a week for 2-12 weeks.

It is another objective of the present invention to provide such methods comprising a continuous dosing schedule having a loading dosing administered intravenously periodically ranging from daily, every other day or 2-5 times a week, once a week, once in two weeks, or once a month for 1-12 weeks.

It is another objective of the present rapid invention to provide such methods wherein the continuous dosing schedule is maintained until the desired rapid therapeutic effect is achieved to treat rapidly, or rapidly prevent, abnormal bone resorption in an osteoporotic mammal, preferably an osteoporotic human.

It is another objective of the present invention to treat rapidly or rapidly prevent fractures in an osteoporotic mammal, preferably an osteoporotic human, and a rapid invention to provide pharmaceutical compositions and kits useful in the methods herein.

These and other objective will become readily apparent from the detailed description, which follows.

Metastatic bone disease involves tumor-induced skeletal metastases, which commonly result from breast cancer, prostate cancer, lung cancer, renal cancer, thyroid cancer, and multiple myeloma. The prevalence of bone-metastases in patients with these cancers may be as high as 60-85%. Patients with these diseases that have bone dominant or bone-only metastases frequently have prolonged survival usually associated with clinical morbidity. The most frequent clinical manifestations of bone metastases are pain, pathological fracture, immobility, nerve root or spinal cord compression, hypercalcemia, and compromised hematopoiesis. The scope of metastatic bone disease is highlighted by the fact that on any given day, approximately 4 million people worldwide suffer from cancer pain and that at least 40-50% of all cancer pain is due to skeletal metastases.

Hypercalcemia of malignancy is also tumor induced. It is characterized by high levels of serum calcium and it may or may not be associated with metastatic bone disease. It is estimated that hypercalcemia develops in 5% to 10% of hospital cancer patients. Symptoms of hypercalcemia include fatigue, malaise, anorexia, polydipsia, nausea, constipation, muscle weakness, apathy, obtundation and even coma. Wimalawansa S. J. Hypercalcaemia of Malignancy Etiology, Pathogenesis and Clinical Management (Medical Intelligence Unit, Monograph), Springer, N.Y. and R. G. Landes Co., Medical Publishers, Austin, Tex., USA, 1995; Wimalawansa S. J. Hypercalcaemia of Malignancy: etiology, pathophysiology, and management. Reviews on Endocrine-Related Cancer, 45: (1993): 5-24, incorporated in entirety. These metabolic complications of malignancy mostly reflect a disseminated disease. Thus, in the majority of cases, malignancy is recognized before the appearance of hypercalcemia. However, in rare situations, such as neuroendocrine tumors, hypercalcemia may run a slower developing course and even precede the discovery of the tumor. Wimalawansa S. J. Combined therapies with calcitonin and corticosteroid, or bisphosphonate for treatment of hypercalcaemia of malignancy. Journal of Bone and Mineral Metabolism, 15: 160-164, 1997.

Many bisphosphonates such as etidronate, clodronate, olpnandronate, alendronate and risedronate are currently administered orally and other bisphosphonates, such as ibandronate, pamidronate, zolondronaic acid, are administered intravenously. The advantage of intravenous therapy over oral dosage therapy is that therapeutically higher levels can be achieved in a relatively short time period. This is especially important in hypercalcemia of malignancy where it is desired to lower serum calcium levels as quickly as possible to minimize calcium deposition and resulting bone disorders.

One of the major problems is that intravenous bisphosphonates are prepared and packaged in glass containers and tend to form a precipitate during shelf storage. This has shown to be a result of metal ion precipitation of a bisphosphonate at increasingly higher pH values forming insoluble metal complexes. Precipitates are dangerous in an intravenous formulation since they can lead to embolisms and blocking of capillaries, which can be fatal. Thus, intravenous formulations must pass a series of rigid government (United States or international) and compendial tests, including the USP (United States Pharmacopeia in the United States) test for particulate matter. With respect to packaging, the use of glass containers is highly desirable since the formulation contents can be quickly inspected for the presence of particulates, and glass is a very efficient and convenient medium for packaging.

What is desired is a therapeutically loading dose of intravenous bisphosphonate formulations, which are solution stable, isotonic with human blood, can be packaged in glass, meet government and compendial (USP in the US) particulate standards, and which can be used as effective therapy to optimally treat patients with metastatic bone disease, hypercalcemia of malignancy or metabolic bone diseases.

### SUMMARY OF THE INVENTION

The present invention relates to methods for rapid inhibition of bone resorption in a mammal in need thereof, a method comprising oral, parenteral, intravenous, intra-arterial administration to said mammal a pharmaceutically effective amount of a loading dose of a bisphosphonate or another bone acting agent a continuous schedule or having a dosing interval selected from the group consisting of once daily, 2-5 times a week, once-weekly, twice-weekly dosing, biweekly dosing, twice-monthly, every few weeks dosing, wherein said continuous schedule is maintained until the desired therapeutic effect is achieved for said mammal. This may be followed up with the standard recommended dose of a bisphosphonate or any suitable agent to maintain or improve BMD and decrease fracture risk (e.g., HRT, SERM or PTH, etc.).

The poor bioavailablity of bisphosphonates means that only about 1% of an orally administered dose is absorbed. This is the case for all the oral agents—alendronate, risedronate, etidronate, tiludronate, etc. Therefore, it generally takes several months or up to a year or more to get an adequate amount of a bisphosphonate incorporated into bone and to have an effect on bone turnover and reduce the risk of fractures.

### DESCRIPTION OF PREFERRED EMBODIMENTS

The methods of this invention is to treat osteoporosis and other bone mineral disorders in menopausal/postmenopausal women and in men who are manifesting the signs and/or symptoms or both (i.e. treatment of osteoporosis) thereof or who are high risk candidates (prevention of osteoporosis) for doing so, e.g., as determined by appropriate clinical conditions. In the case of a male, for both primary and secondary osteoporosis, an added effect is achieved when a loading dose of a bisphosphonate is administered concurrently with an androgen or an androgenic analogue. Thus, the method aspect of this invention, the loading dose of a bisphosphonate with or without any other bone active agent such as PTH, calcitonin, SERMs, vitamin D and other analogues, an androgen, nitroglycerin, growth factors, or other agents that affect bone metabolism.

In several embodiments, the present invention relates to methods comprising a rapid, but continuous dosing schedule having a dosing periodicity ranging from once daily to once a week or otherwise.

In other embodiments, the present invention relates to methods for rapidly treating abnormal bone resorption in a mammal in need of such treatment.

In other embodiments, the present invention relates to methods for rapidly preventing abnormal bone resorption in a mammal in need of such prevention.

In other embodiments, the present invention relates to such methods useful in humans.

In other embodiments, the present invention relates to methods for treating or preventing osteoporosis in a mammal.

In other embodiments, the present invention relates to such methods useful in treatment of osteoporosis and Paget's disease of bone in humans.

In other embodiments, the present invention relates to such methods useful in humans identified as having bone prosthesis, metabolic bone diseases, or metastatic bone disease.

In other embodiments, the present invention relates to methods for treating or preventing osteoporosis-associated fractures and controlling pain in a human.

In other embodiments, the present invention relates to methods for treating pain associated with any bone disease or fractures in a human.

In other embodiments, the present invention relates to methods for prevention of bone loss associated with microgravity, bed-ridden conditions such as following stokes or a fracture, or for astronauts in long-duration space flights.

In other embodiments, the present invention relates to methods for rapidly inhibiting bone resorption or treating or rapidly preventing abnormal bone resorption in a human comprising administering to a said human.

### FIELD OF THE INVENTION

The present invention relates to either oral, parenteral or intravenous administration of a loading dose of a bisphosphonate or any other bone active agent for rapid inhibition of bone resorption or enhanced bone formation or both, in a mammal. These methods comprise orally or intravenously administering to a mammal in need thereof of a pharmaceutically effective amount of a bisphosphonate or other bone acting agents as a loading does at the onset of the treatment regimen as a unit dosage according or a continuous schedule dosing. The present invention also relates to pharmaceutical compositions and kits useful for carrying out these methods.

### DESCRIPTION OF THE INVENTION

The present invention relates to a method; preferably an oral or intravenous method for rapid inhibiting bone resorption in a mammal. The present invention relates to methods of treating or preventing abnormal or rapid bone resorption in a mammal in need of such treatment or prevention. The methods of the present invention comprise orally, parenterally, or intravenously administering to a mammal a pharmaceutically effective amount of a loading dose of a bisphosphonate as a unit dosage, wherein said dosage is administered according to a continuous schedule having a loading dose interval selected from the group consisting of daily, several days a week, or weekly dosing for several weeks. Typically, the continuous dosing schedule is maintained until the desired therapeutic effect is achieved for the mammal.

The methods of the present invention are generally administered to mammals in need of a loading dose of a bisphosphonate therapy. Preferably the mammals are human patients, particularly human patients in need of rapid inhibition of bone resorption, such as patients in need of treating or preventing abnormal bone resorption.

The term “pharmaceutically effective amount”, as used herein, means that amount of the bisphosphonate compound, which will elicit the desired therapeutic effect or response when administered in accordance with the desired treatment regimen. A preferred pharmaceutically effective amount of the bisphosphonate is a bone resorption-inhibiting amount, as measured by biomarkers or bone biopsy, BMD, micro-computerized tomography (micro-CT), peripheral-CT, or another technique.

The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure as well.

The term “bone resorption inhibition”, as used herein, means treating or preventing bone resorption by the direct or indirect alteration of osteoclast formation or activity. Inhibition of bone resorption refers to treatment or prevention of bone loss, especially the inhibition of removal of existing bone either from the mineral phase and/or the organic matrix phase, through direct or indirect alteration of osteoclast formation or activity.

The terms “continuous schedule” or “continuous dosing schedule”, as used herein, mean that the dosing regimen is repeated until the desired therapeutic effect is achieved. The continuous schedule or continuous dosing schedule is distinguished from cyclical or intermittent administration.

The term “until the desired therapeutic effect is achieved”, as used herein, means that the bisphosphonate compound is continuously administered, according to the dosing schedule chosen, up to the time that the clinical or medical effect sought for the disease or condition is observed by the clinician or researcher. For methods of treatment of the present invention, a loading dose of a bisphosphonate compound is administered until the desired change in biomarkers or bone mass or structure is observed. In such instances, achieving an increase in bone density, decrease in biomarkers, or a replacement of abnormal bone structure with more normal bone structure are the desired objectives.

For methods of prevention of the present invention, the bisphosphonate compound is continuously administered for as long as necessary to prevent the undesired condition. In such instances, maintenance of bone density and prevent fractures are often the objective. For humans, administration periods can range from daily to once a week to the remaining lifespan of the human.

### METHODS OF THE PRESENT INVENTION

The present invention comprises methods for rapidly inhibiting bone resorption in mammals. The present invention also comprises rapidly treating abnormal bone resorption in mammals and expedited fracture reduction. The present invention also comprises methods for rapidly preventing abnormal bone resorption in mammals. In preferred embodiments of the present invention, the mammal is a human, but this is also applicable to other mammals as well. Also continuous or a loading dosing schedule whereby a unit dosage of a higher amount of the bisphosphonate is administered to a mammal.

In further embodiments or descriptions of the present invention, the unit dosage is loading doses of bisphosphonate given with a periodicity ranging from daily to several days a week, to several weeks by oral or intravenously administered bisphosphonates.

The methods and compositions of the present invention are useful for rapidly inhibiting bone resorption and for rapidly treating and preventing abnormal bone resorption and conditions associated therewith. Such conditions include both generalized and localized bone losses. Also, the creation of bone having an abnormal structure, as in Paget's disease, can be associated with abnormal bone resorption. The term “generalized bone loss” means bone loss at multiple skeletal sites or throughout the skeletal system. The term “localized bone loss” means bone loss at one or more specific, defined skeletal sites as in the case of rheumatoid arthritis or Sudex's atrophy.

Generalized boss loss is often associated with osteoporosis. Osteoporosis is most common in postmenopausal women, wherein estrogen production has been greatly diminished. However, osteoporosis can also be glucocorticoid-induced and has also been evident in males due to aging. Osteoporosis can be induced by disease, e.g. rheumatoid arthritis, it can be induced by secondary causes, e.g., glucocorticoid therapy, or it can come about with no identifiable cause, i.e., idiopathic osteoporosis. In the present invention, preferred methods include the rapidly decreasing abnormal bone resorption in osteoporotic humans or anyone with metabolic bone diseases or metastatic bone diseases.

Localized bone loss has been associated with periodontal disease, with bone fractures, and with periprosthetic osteolysis (i.e., where bone resorption has occurred in proximity to a prosthetic implant). Generalized or localized bone loss can occur from disuse, which is often a problem for those confined to a bed or a wheelchair, or for those who have an immobilized limb set in a cast or in traction.

This method can be also use to prevent bone losses associated with microgravity, bed-ridden conditions such as following a stoke or a fracture, or for astronauts in long-duration space flights.

The methods and compositions of the present invention are useful for treating and or preventing the following conditions or disease states: osteoporosis, which can include postmenopausal osteoporosis, glucocorticoid-induced osteoporosis, male osteoporosis, disease-induced osteoporosis, idiopathic osteoporosis; Paget's disease; abnormally increased bone turnover; periodontal disease; localized bone loss associated with periprosthetic osteolysis; and bone fractures, etc.

The methods of the present invention are intended to specifically exclude methods for the treatment and/or prevention of prosthesis loosening and prosthesis migration in mammals as described in PCT application WO 95/30421, to Goodship et al, published Nov. 16, 1995, which is incorporated by reference herein in its entirety.

Bisphosphonates

The methods and compositions of the present invention comprise a bisphosphonate. The bisphosphonates of the present invention correspond to the chemical formula ##STR1##

Wherein

A and X are independently selected from the group consisting of H, OH, halogen, NH.sub.2, SH, phenyl, C1-C30 alkyl, C1-C30 substituted alkyl, C1-C10 alkyl or dialkyl substituted NH.sub.2, C1-C10 alkoxy, C1-C10 alkyl or phenyl substituted thio, C1-C10 alkyl substituted phenyl, pyridyl, furanyl, pyrrolidinyl, imidazonyl, and benzyl.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH.sub.2, C1-C10 alkyl or dialkyl substituted NH.sub.2, OH, SH, and C1-C10 alkoxy.

In the foregoing chemical formula, A can include X and X can include A such that the two moieties can form part of the same cyclic structure.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Preferred structures are those in which A is selected from the group consisting of H, OH, and halogen, and X is selected from the group consisting of C1-C30 alkyl, C1-C30 substituted alkyl, halogen, and C1-C10 alkyl or phenyl substituted thio.

More preferred structures are those in which A is selected from the group consisting of H, OH, and Cl, and X is selected from the group consisting of C1-C30 alkyl, C1-C30 substituted alkyl, Cl, and chlorophenylthio.

Most preferred is when A is OH and X is a 3-aminopropyl moiety, so that the resulting compound is a 4-amino-1,-hydroxybutylidene-1,1-bisphosphonate, i.e. alendronate, or risedronate.

Pharmaceutically acceptable salts or an acid or any other compound and derivatives of the bisphosphonates are also useful herein. Non-limiting examples of salts include those selected from the group consisting of alkali metal, alkaline metal, ammonium, and mono-, di, tri-, or tetra-C1-C30-alkyl-substituted ammonium.

Preferred salts are those selected from the group consisting of sodium, potassium, calcium, magnesium, and ammonium salts. Non-limiting examples of derivatives include those selected from the group consisting of esters, hydrates, and amides.

“Pharmaceutically acceptable” as used herein means that the salts and derivatives of the bisphosphonates have the same general pharmacological properties as the free acid form from which they are derived and are acceptable from a toxicity viewpoint.

It should be noted that the terms “bisphosphonate” and “bisphosphonates”, as used herein in referring to the therapeutic agents of the present invention, are meant to also encompass diphosphonates, biphosphonic acids, and diphosphonic acids, as well as salts or ester, and derivatives of these materials. The use of a specific nomenclature in referring to the bisphosphonate or bisphosphonates is not meant to limit the scope of the present invention, unless specifically indicated. Because of the mixed nomenclature currently in use by those or ordinary skill in the art, reference to a specific weight or percentage of a bisphosphonate compound in the present invention is on an acid active weight basis, unless indicated otherwise herein.

Nonlimiting examples of bisphosphonates useful herein include the following:

Alendronic acid, 4-amino-1-hydroxybutylidene-1,1-bisphosphonic acid.

Alendronate (also known as alendronate sodium or monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990, and 5,019,651, to Kieczykowski, issued May 28, 1991, both of which are incorporated by reference herein in their entirety.

Cycloheptylaminomethylene-1,1-bisphosphonic acid, YM 175, Yamanouchi (cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1 -bisphosphonic acid (risedronate).

(4-chlorophenyl)thiomethane-1,1-disphosphonic acid (tiludronate) as described in U.S. Pat. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zolendronate).

Any other bisphosphonate.

Preferred are bisphosphonates selected from the group consisting of alendronate, cimadronate, clodronate, tiludronate, etidronate, olpandronate, ibandronate, risedronate, piridronate, pamidronate, zolendronate (zolondronic acid), or any newer bisphosphonate in a pharmaceutically acceptable salt, or an acid thereof, and mixtures thereof.

Pharmaceutical Compositions

Compositions useful in the present invention comprise a pharmaceutically effective amount of a bisphosphonate. The bisphosphonate is typically administered in a mixture with suitable pharmaceutical diluents, excipients, or carriers, collectively referred to herein as “carrier materials”, suitably selected with respect to oral administration, i.e., tablets, capsules, elixirs, syrups, effervescent compositions, powders, and the like, and consistent with conventional pharmaceutical practices. For example, for oral administration in the form of a tablet, capsule, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs and syrups, effervescent compositions, the oral drug components can be combined with any oral, non-toxic, pharmaceutically acceptable inert carrier such as ethanol, glycerol, water and the like. Moreover, when desired or necessary, suitable binders, lubricants, disintegrating agents, buffers, coatings, and coloring agents can also be incorporated.

Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. A particularly preferred tablet formulation for an oral bisphosphonate is that described in U.S. Pat. No. 5,358,941, to Bechard et al, issued Oct. 25, 1994, which is incorporated by reference herein in its entirety. The compounds used in the present method can also be coupled with soluble polymers as targetable drug carriers. Such polymers can include polyvinylpyrrolidone, pyran copolymer, polyhydroxylpropylmethacrylamide, and the like.

The precise loading dosage of the bisphosphonate required will vary with the potency of the individual bisphosphonate and the individual patient requirement. The oral potency of a particular bisphosphonate that is chosen also depends on the age, size, sex and condition of the mammal or human, the nature and severity of the disorder to be treated, and other relevant medical and physical factors.

Thus, a precise pharmaceutically effective dose of a given agent cannot be specified in advance and can be readily determined by the caregiver or clinician. Appropriate amounts can be determined by routine experimentation from animal models and human clinical studies. Generally, an appropriate amount of a loading dose of a bisphosphonate is chosen to obtain a rapid bone resorption inhibiting effect, i.e. a bone resorption inhibiting amount of the bisphosphonate is administered as a loading dose. For humans, an effective oral dose of bisphosphonate is typically about 1.0 to about 10,000 μg/kg body weight and preferably about 5 to 5,000 μg/kg of body weight.

Non-limiting examples of oral compositions comprising alendronate and risedronate, as well as other bisphosphonates, are illustrated in the Examples, below.

Treatment Kits

In further embodiments, the present invention relates to a kit for conveniently and effectively carrying out the methods in accordance with the present invention. Such kits are especially suited for the delivery of solid oral forms such as tablets or capsules. Such a kit preferably includes a number of unit dosages, including tables or liquid form. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered. Alternatively, placebo dosages, or calcium or dietary supplements, either in a form similar to or distinct from the bisphosphonate dosages, can be included to provide a kit in which a dosage is taken every day.

Oral Bisphosphonates:

Bisphosphonate containing tablets are prepared using standard mixing and formation techniques as described in U.S. Pat. No. 5,358,941, to Bechard et al., issued Oct. 25, 1994, which is incorporated by reference herein in its entirety. Tablets containing about 70 mg of alendronate, or 35 mg of risedronate, which are FDA approved to be used for once a week, are already available to be used as loading doses (i.e., daily administration).

The FDA approved doses of oral bisphosphonates are 40 mg and 70 mg tables for alendronate, and 30 and 35 mg tablets for risedronate. These doses are expected to rapidly decrease the bone turnover within days of administration instead of weeks or months. The present invention allows achieving this rapid response, which is required in many patients to prevent a new fracture or subsequent fractures. These bisphosphonates or other similar compounds can also be administer in addition to tablet forms, as liquid forms to minimize the gastric irritation and to handle the larger doses necessary for the front loading of these agents. Further advantage of these liquid forms would be to titrate the doses easier than tablets (e.g., administration of 30-400 mg of alendronate or risedronate as a oral elixir or other liquid forms).

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### BRIEF DESCRIPTION OF THE DRAWINGS

### Example 1

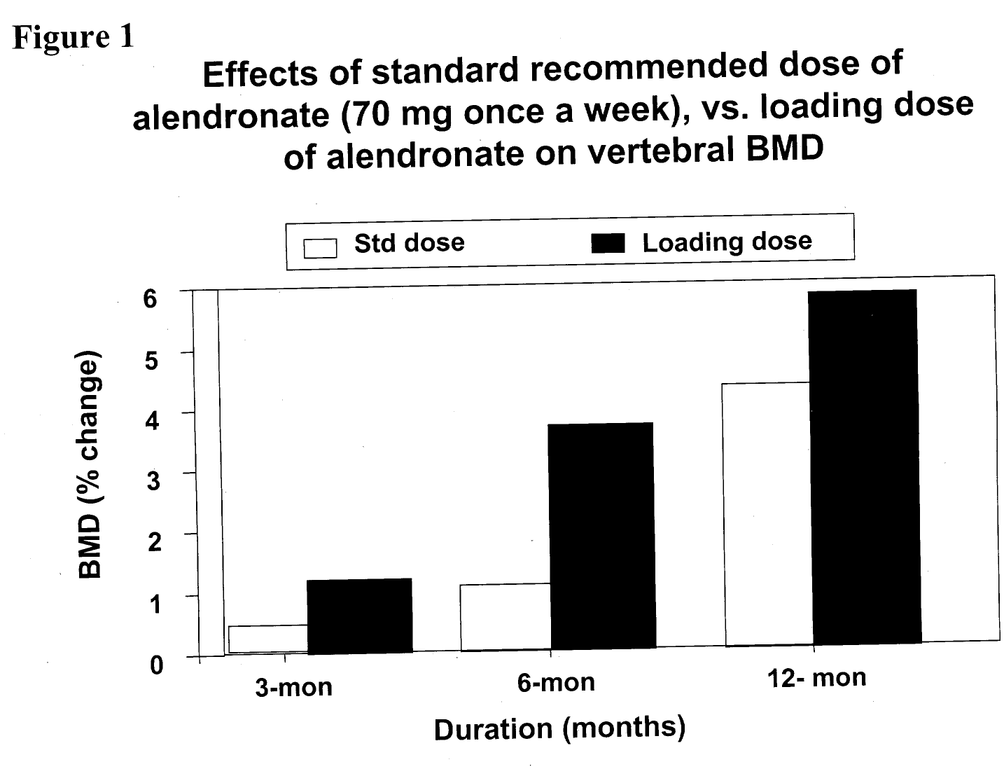

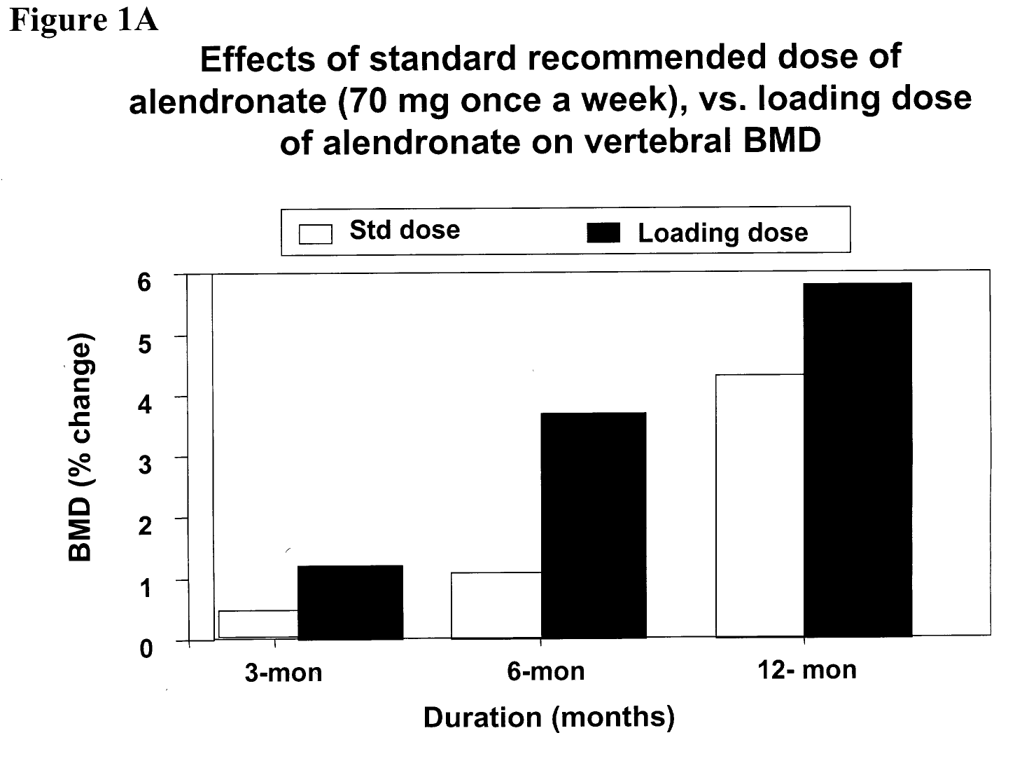

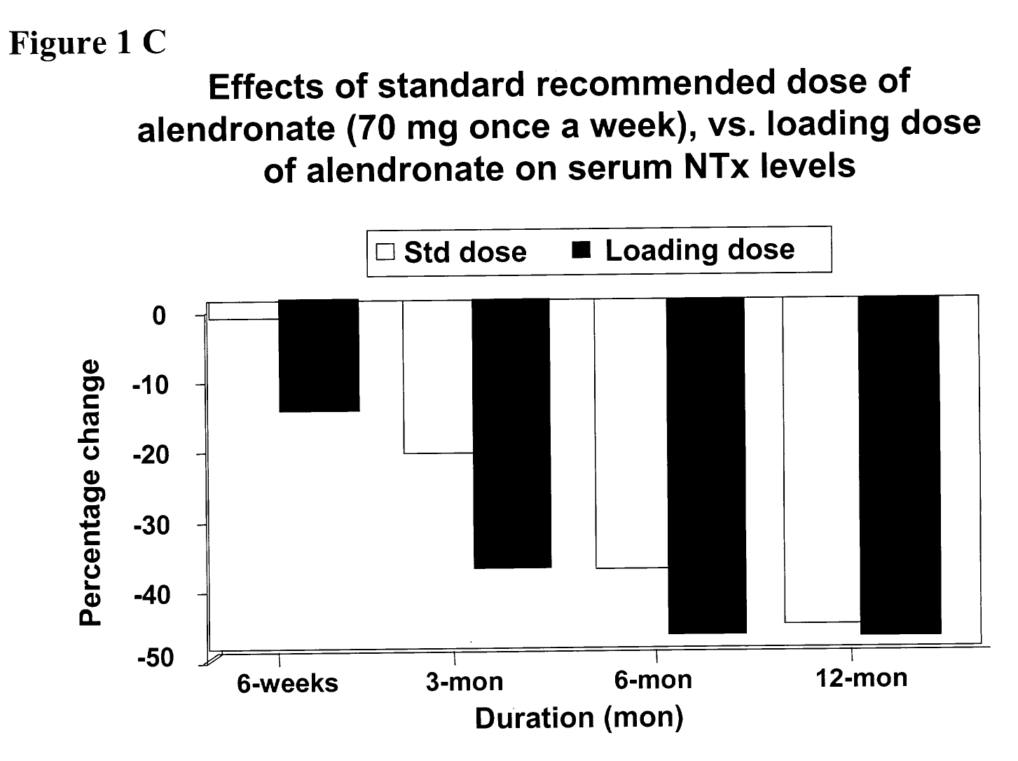

FIG. 1 illustrates the effects of standard recommended dose of alendronate (70 mg once a week), vs. loading dose of alendronate (for doses, see the section on oral administration of bisphosphonates) on vertebral bone mineral density (BMD).

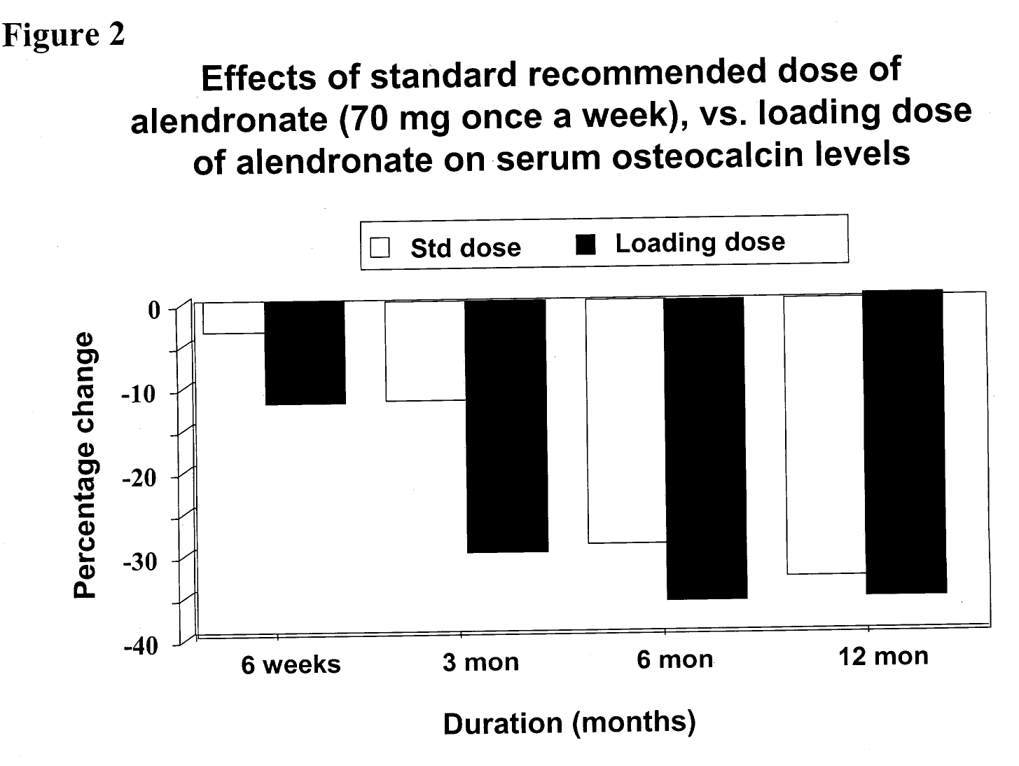

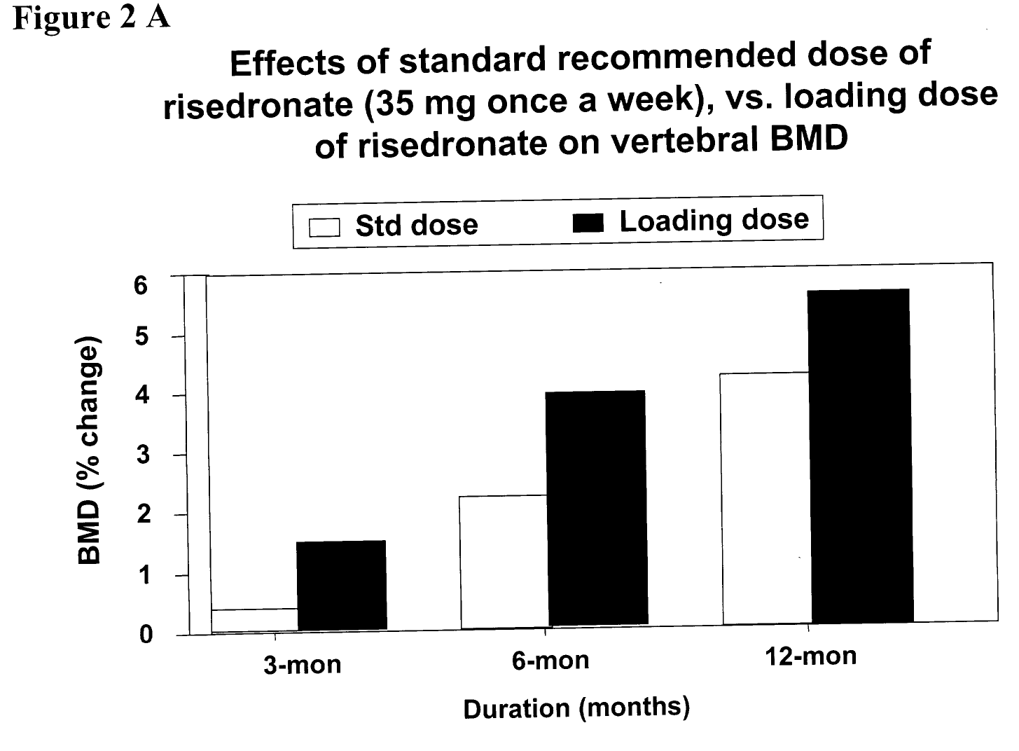

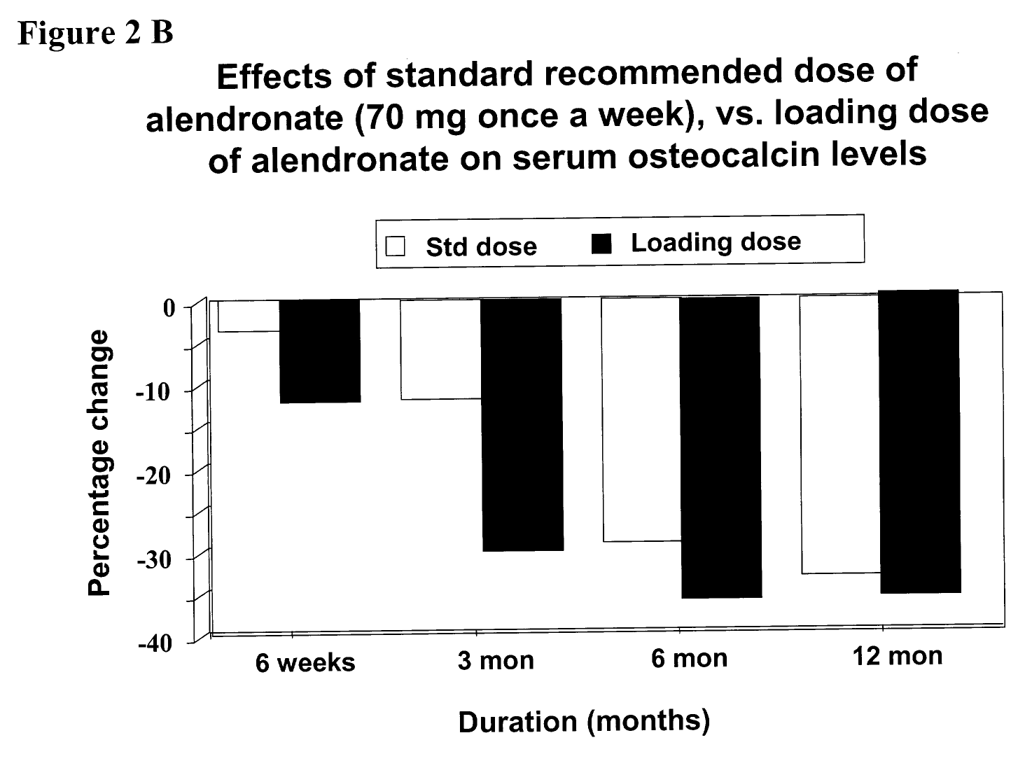

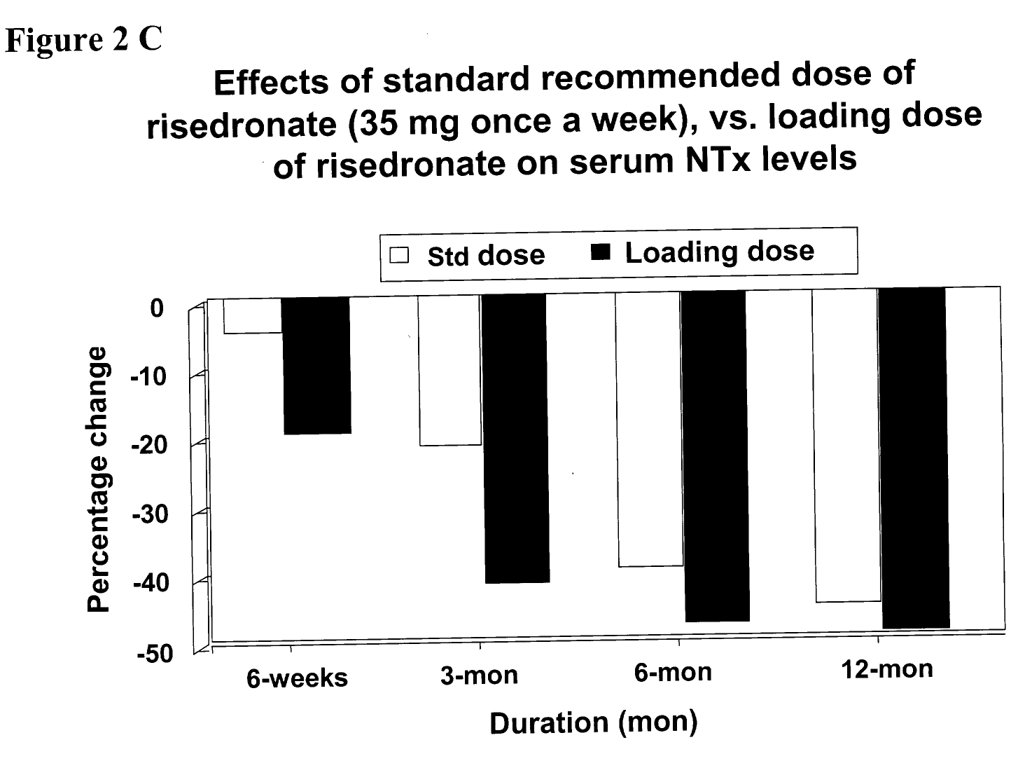

FIG. 2 illustrates the effects of standard recommended dose of alendronate (70 mg once a week), vs. loading dose of alendronate on bone formation marker, serum osteocalcin levels.

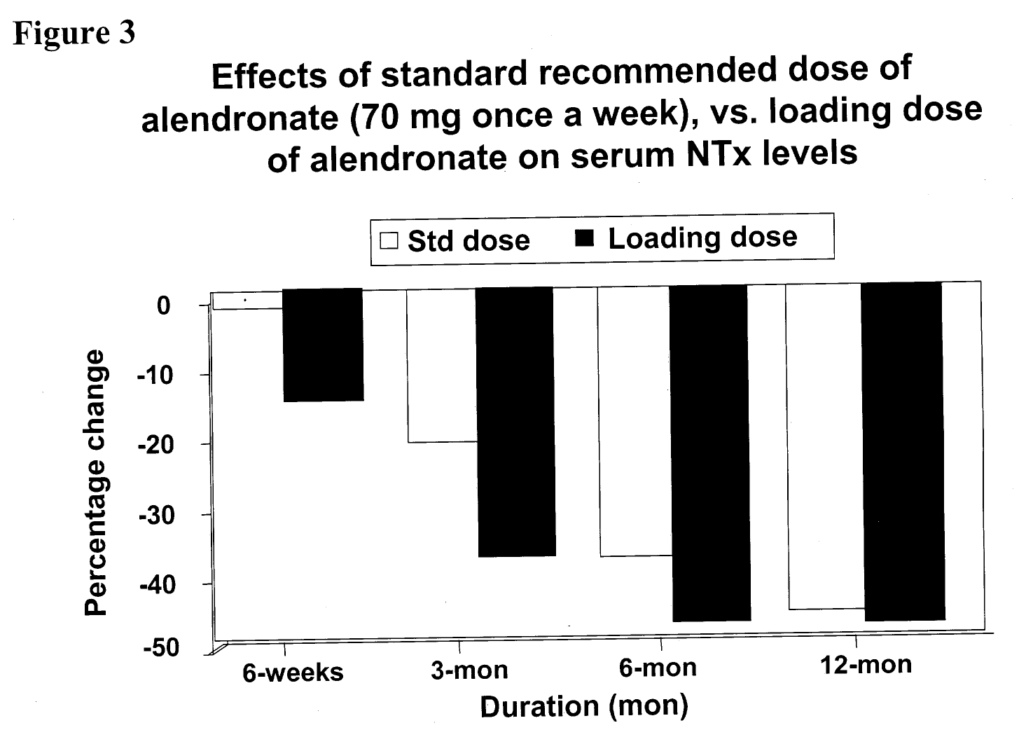

FIG. 3 illustrates the effects of standard recommended dose of alendronate (70 mg once a week), vs. loading dose of alendronate on bore resorption marker, serum NTx levels.

Comparison of BMD increases with conventional
and loading doses of alendronate:

| Increase in spinal BMD | Standard dose | Loading dose |
|---|---|---|
| 3 months | +0.4% | +1.2% |
| 6 months | +1.1% | +3.6% |
| 12 months | +4.5% | +5.8% |

### Example 2

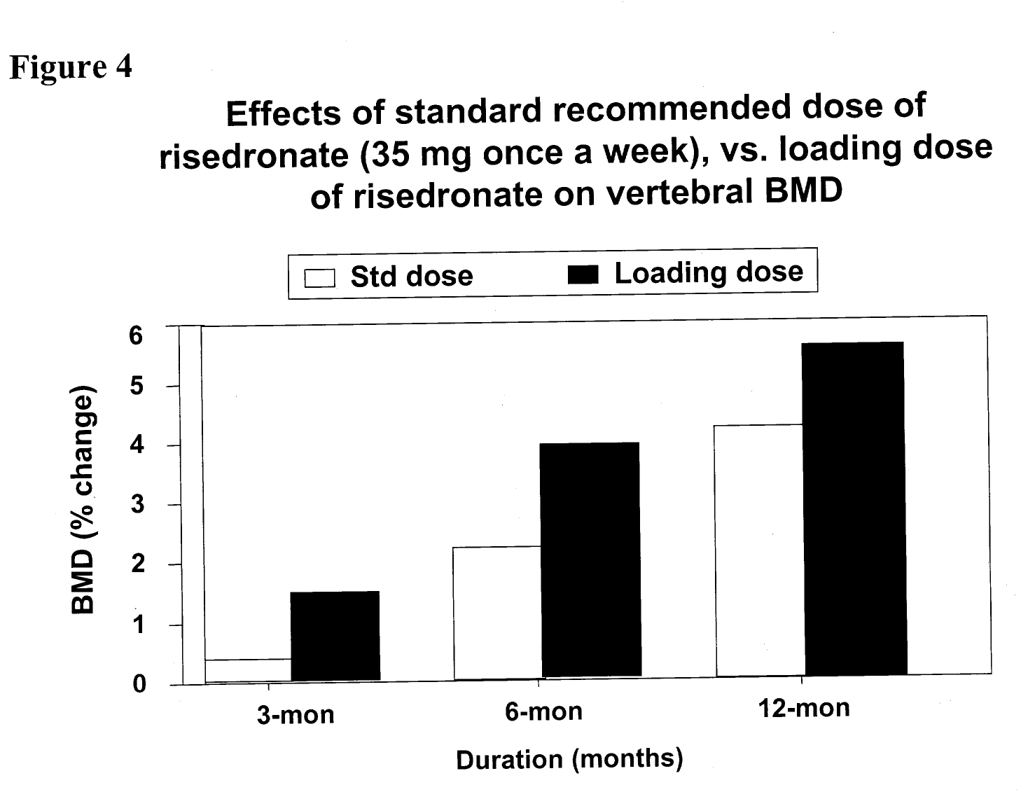

FIG. 4. illustrates the effects of standard recommended dose of risedronate (35 mg once a week), vs. loading dose of risedronate (for doses, see the section on oral administration of bisphosphonates) on vertebral BMD.

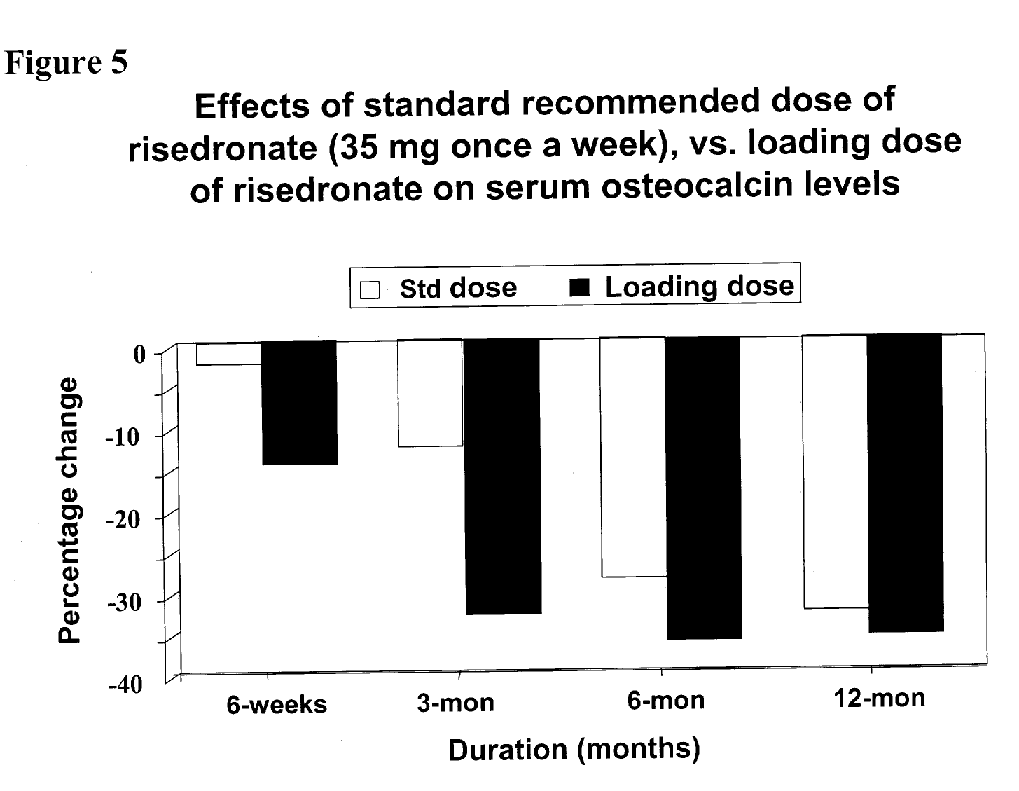

FIG. 5. illustrates the effects of standard recommended dose of risedronate (35 mg once a week), vs. loading dose of risedronate on bone formation marker, serum osteocalcin levels.

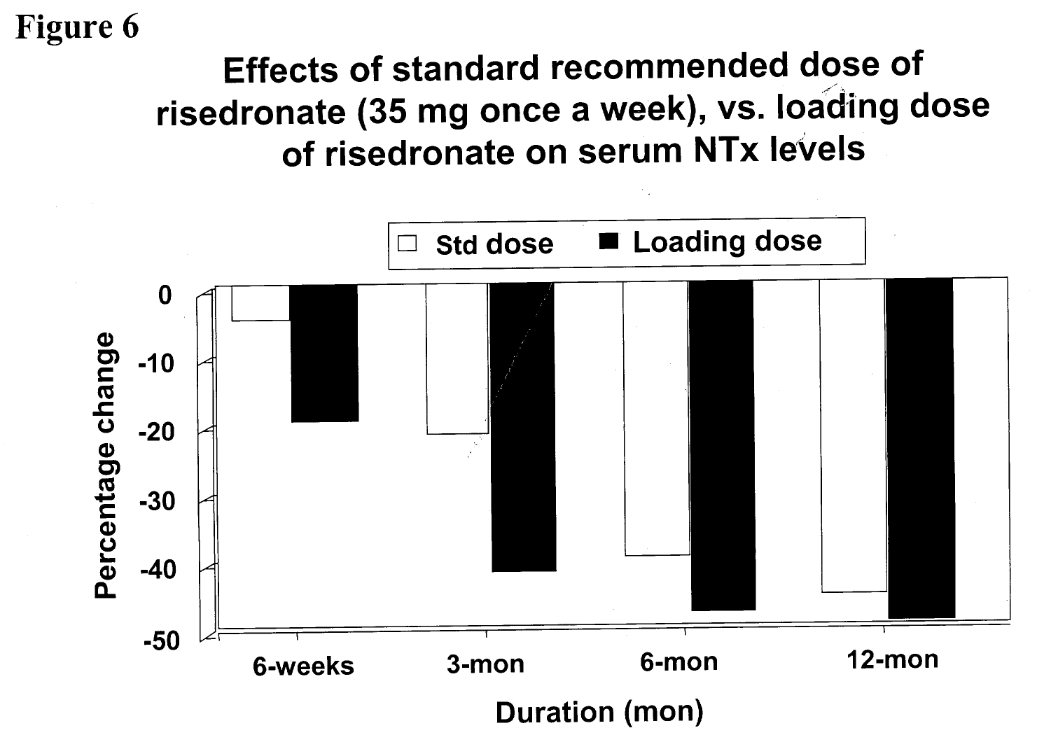

FIG. 6. illustrates the effects of standard recommended dose of risedronate (35 mg once a week), vs. loading dose of risedronate on bone resorption marker, serum NTx levels.

Comparison of BMD increases with conventional
and loading doses of risedronate:

| Increase in spinal BMD | Standard dose | Loading dose |
|---|---|---|
| 3 months | +0.4% | +1.5% |
| 6 months | +2.2% | +4.0% |
| 12 months | +4.3% | +5.6% |

In addition, there were more rapid changes in the biochemical markers of bone turnover—seen within 4-8 weeks with loading doses, compared with the conventional dosing.

### Example 3

Prevention and/or Treatment of Osteoporosis and Rapid Fracture Reduction.

Oral Administration of Bisphosphonates:

Alendronate (70 mg) or risedronate tablets (35 mg) (or liquid formulations containing about 40 or 70 mg of alendronate or 30-50 mg risedronate) are orally administered to a humans daily for 1-6 weeks (preferably 2-3 weeks) duration; i.e., the loading dose of either alendronate or risedronate or any other bisphosphonates. This will then be followed up with once weekly administration (i.e., infrequent administration) of a single dose of similar strength, what is currently used on a longer-term basis. This method of administration is not only useful and convenient, but also able to rapidly decrease bone turnover and fracture rates in osteoporosis and other bone diseases.

Getting the drug into the bone earlier should result in a more rapid response and so enhance the beneficial effects on BMD and fracture reduction. The loading dose regimen should result in a more rapid response. After both oral and intravenous administration there was a significant increase in BMD within 3-6 months with the loading doses, compared to 9-12 months with conventional therapy.

### Example 4

Intravenous Administration of Bisphosphonates:

Pamidronate, Ibandronate, Zolendronate, and other newer bisphosphonates liquid formulations can be administered intravenously. The liquid formulations are administered to a human patient daily, several days a week, once or twice weekly, preferably about once every three or four days (for example, every Monday, Wednesday and Friday), for a period of 1-6 weeks. For example, 90 mg pamidronate will be administered in one liter of normal saline over 5 hours as an intravenous infusion into a large vein. This will be repeated (i.e., 90 mg) one or two more times (i.e., a total dose of 180-270 mg), infusions given after 2-5 days, or 1-4 weeks apart (the loading dose). This will be followed up with 90 mg of pamidronate administered once in 6 months or in some instances once a year, as monitored by biomarker responses. In the case of ibandronate 4 mg is administered intravenously once a week for 2-4 weeks. This will be followed up with the standard dose of 2-4 mg administered every 3-4 months, or higher doses administered infrequently. In the case with zolondronic acid (zolendronate), initial two doses (loading doses) of 3-5 mg is each administered 1-4 week apart as a slow intravenous infusion over a 15-minute period. This will then be followed up with the standard doses of 4-5 mg administered once a year. The doses and the frequencies illustrated here are for examples only and can be varied depending on the circumstances. Other formulations of parenteral and intravenous formulations of all bisphosphonates can be administered in a similar fashion to enhance its efficacy in fracture reduction. This method of administration is useful and convenient for treating osteoporosis and for minimizing adverse gastrointestinal effects, particularly adverse esophageal effects. This method is also useful for improving patient acceptance and compliance, and more importantly rapid fracture reduction.

What is claimed:

1. A method comprising at least one pharmaceutically effective dosage of a bisphosphonate selected from the group consisting of alendronate, cimadronate, clodronate, tiludronate, etidronate, ibandronate, risedronate, piridronate, olpandronate, pamidronate, zoledronate or any other new bisphosphonates in a pharmaceutically acceptable salts, acid, or esters thereof, and mixtures thereof, for oral tablets or liquid form or intravenous administration, or administration via other parenteral means to a mammal having a loading dosing interval selected depending on the potency of the agent.

2. A kit comprising at least one pharmaceutically effective dosage of a bisphosphonate selected from the group consisting of alendronate, cimadronate, clodronate, tiludronate, etidronate, ibandronate, risedronate, piridronate, olpandronate, pamidronate, zoledronate, or any other new bisphosphonates in a pharmaceutically acceptable salts, acid, or esters thereof, and mixtures thereof, for oral tablets or liquid form or intravenous administration, or administration via other parenteral means to a mammal, preferably a human male or female, having a loading dosing interval selected depending on the potency of the agent.

3. A method of claims 1 and 2, where a loading dose of a bisphosphonate or an another bone acting agent is administered orally tablets or liquid form (daily, several days a week, weekly, etc.), or the agent is administered intravenously or another parenteral route as a loading dose to rapidly reduce bone turnover, increase BMD and decrease fracture rates.

4. A method of claims 1 through 3 for prevention or treatment of primary and secondary osteoporosis of a female or male mammal comprising administering to an afflicted mammal, or one in need thereof, loading dose of oral tablets or liquid form or parenterally administered bisphosphonate by itself.

5. A method claims 1 through 3, for prevention or treatment of primary and secondary osteoporosis of a female or male mammal comprising administering to an afflicted mammal, or one in need thereof, loading dose of a bisphosphonate together with another agent acting on bone, including any estrogenic compound administered continuously or cyclically with or without a progestin agent (progesterone, dyprogesterone, medroxyprogesterone, norethisterone, levonorgestrel, or norgestrel, etc.) or both; tibolone; any androgen, testosterone or testosterone analogue; anabolic steroid (nandrolone decanoate); estrogen receptor modulating agent SERMs; PTH or its analogues; any calcitonin; vitamin D or its analogues; bone active growth factors, peptides or proteins, or their analogues; fluoride (fluoride salt is a rapid or slow release sodium fluoride formulation); an nitric oxide donor, it's substrate, or a pharmacologically acceptable nitrate, any of these agents administered continuously, intermittently, or cyclically.

6. The method of claims 1 through 3, wherein the mammal is a female or male human suffering from primary or secondary osteoporosis, and/or preventing or alleviating pain associated with osteoporosis.

7. The method of claims 1 through 3, wherein the mammal is a human and the treatment further comprises a loading dose of a bisphosphonate is administered at least with calcitonin, HRT, SERMs, nitroglycerin, PTH, fluoride, or anabolic steroid, administered continuously or cyclically.

8. The method of claims 1 through 3, a pharmaceutical composition comprising synergistic effective amounts of a loading dose of a bisphosphonate, is administered with other bone-acting compound.

9. A method of preventing and treating any metabolic bone diseases in a mammal preferably human, comprising administering to the subject a loading dose of a bone resorption inhibiting agent (e.g., a bisphosphonate), or a bone stimulating agent (i.e., anabolic agent), as a pharmaceutically acceptable salt, acid or thereof, or another bone-acting agent either orally or parenteralily including intravenously administered bisphosphonate or a pharmaceutically acceptable salt, acid, or thereof.

10. A method of preventing and treating any metastatic bone disease in a mammal, preferably a human, administering to the subject a bone resorption inhibiting amount of a loading dose of an oral tablets or liquid form, or parenterally or intravenously administered bisphosphonate as a pharmaceutically acceptable salt, acid, or thereof.

11. A method of preventing or alleviating pain associated with any metabolic bone disease, crush fracture syndrome, metastatic bone disease, or Paget's disease of bone in a mammal, preferably human, using administering a bone resorption inhibiting amount of a loading dose of an oral tablets or liquid form, intravenously or intra arterially administered bisphosphonate as a pharmaceutically acceptable salt, acid, or thereof.

12. A method of treating or preventing recurrences of Paget's disease as well as pain associated with the Paget's disease of bone in a mammal, preferably human, using administering a bone resorption inhibiting amount of a loading dose of an oral, parenterally, or intravenously administered bisphosphonate as a pharmaceutically acceptable salt, acid, or thereof.

13. A method of preventing bone losses associated with microgravity, bed-ridden conditions such as following stokes or a fracture, or for astronauts in long-duration space flights using administering a bone resorption inhibiting amount of a loading dose of an oral, intravenously or intra arterially (parenteral) as a pharmaceutically acceptable bisphosphonate salt, acid, or thereof.

14. A method in claims 1 though 3, wherein said unit dosage of said bisphosphonate kits comprise about 1.0 to 6000 μ/kg body weight, or the unit dosage of said bisphosphonate comprises about 5 to 2000 μg/kg body weight.

15. A kit according to claim 2 wherein said bisphosphonate is selected from the group consisting of alendronate, risedronate, zolendronate, cimadronate, clodronate, tiludronate, etidronate, ibandronate, piridronate, olpandronate, pamidronate, or any other new bisphosphonates in a pharmaceutically acceptable salt, acid, or an esters thereof, and mixtures thereof.

16. A kit according to claim 2 and 15, wherein said pharmaceutically acceptable salt is selected from the group (e.g., of any bisphosphonate) consisting of sodium, potassium, calcium, magnesium, and ammonium salt or an acid, or esters thereof, and mixtures thereof.

17. A kit according to claim 2 comprising at least one pharmaceutically effective unit dosage of a loading doses of an orally tablets or liquid form, parenterally, or intravenously administered bisphosphonate selected from the group consisting of alendronate, cimadronate, clodronate, tiludronate, etidronate, risedronate, ibandronate, zolendronate, olpandronate, piridronate, pamidronate, or any other bisphosphonate agent, pharmaceutically acceptable salts, acid, or esters thereof, and mixtures thereof, for administration to a mammal according to a continuous or intermittent schedule.

18. A kit according to claim 2 wherein said unit dosage of a kit comprises a memory aid form in a tablet or capsule, a blister pack, unit dosages and are oriented in the order of their intended use.

19. Loading dose of bisphosphonate therapy in patients who have an orthopedic implant device and a method of retarding loosening of an orthopedic prosthesis in a subject for treating and for preventing peri-prosthetic bone loss and/or pain by the administration of a loading dose of an oral, intravenous, or parenterally administered bisphosphonate, or an anabolic agent, or another bone acting agent in by itself or in combination with a bisphosphonate of a pharmaceutically acceptable salt, acid or, thereof, to decrease bone resorption and fracture reduction.

20. Loading dose of bisphosphonate therapy for treating, retarding, or preventing transplant- or drug-induced bone loss and/or bone pain by the administration of an oral tablets or liquid form, or parenterally administered bisphosphonate, or an anabolic agent, or another bone acting agent by itself or in combination with a bisphosphonate of a pharmaceutically acceptable salt, acid or, thereof, to decrease bone resorption and fracture reduction.
